(19) United States
(12) Patent Application Publication
(10) Pub. No.: US 2005/0196466 A1
(43) Pub. Date: Sep. 8, 2005

(54) Process for inhibiting cell proliferation

(75) Inventor: Darren Magda, Cupertino, CA (US)

Correspondence Address: Vinit G. Kathardekar;Pharmacyclics, Inc., 995 E. Arques Avenue, Sunnyvale, CA, 94085, US

(73) Assignee: Pharmacyclics, Inc., Sunnyvale, CA (US)

(21) Appl. No.: 11/040,542

(22) Filed: Jan. 19, 2005

Related U.S. Application Data
(60) Provisional application No. 60/549,324, filed on Mar. 2, 2004.

(51) Int. Cl. A61K 31/675; A61K 31/555; A61K 33/32

(52) U.S. Cl. 424/641; 514/185; 514/410

(57) ABSTRACT

The present invention relates to a process of inhibiting proliferation of neoplastic cells in a host, said process comprising administering to the host: (a) a zinc reagent; and
(b) a compound of Formula I 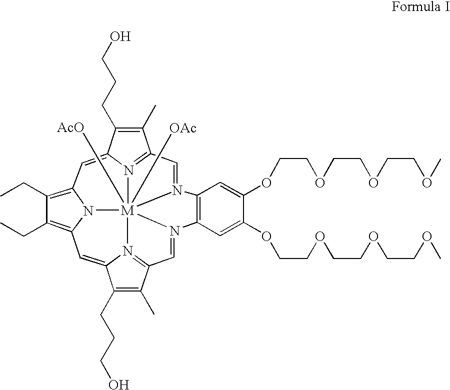
wherein M is selected from Gd⁺⁺⁺, Eu⁺⁺⁺, Tb⁺⁺⁺, Dy⁺⁺, Ho⁺⁺⁺ and Er⁺⁺⁺.

### CLAIM OF PRIORITY

The present application claims the benefit of priority from U.S. Provisional Application Ser. No. 60/549,324, filed Mar. 2, 2004, the contents of which are incorporated herein by reference in its entirety.

### FIELD OF INVENTION

The present invention relates to a process of inhibiting proliferation of neoplastic cells in a host.

### BACKGROUND OF THE INVENTION

It has been reported that compounds of Formula I selectively localize in tumors and promote stress by oxidizing intracellular reducing species. It was recently shown by microarray analysis that treatment of A549 human lung carcinoma cells with MGd led to induction of metallothioneins (MT) and zinc transporter 1 (Hacia, Proc. AACR 43:3211, 2002). It has also been reported that compounds of Formula I catalytically oxidize vicinal thiols such as those present in the active site of thioredoxin reductase and the cofactor dihydrolipoate (Biaglow, Proc. AACR 42:3589, 2001).

None of the research however suggests that compounds of Formula I have an anti-proliferative effect on neoplastic cells. It has been surprisingly found that administering a compound of Formula I and a source of zinc inhibits proliferation of neoplastic cells in a host.

### SUMMARY OF THE INVENTION

The present invention relates to a process for inhibiting proliferation of neoplastic cells in a host, said process comprising administering to a host:
(a) a zinc reagent; and
(b) a compound of Formula I 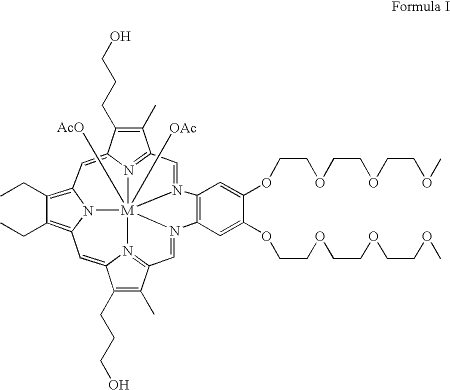
   wherein M is selected from Gd⁺⁺⁺, Eu⁺⁺⁺, Tb⁺⁺⁺, Dy⁺⁺, Ho⁺⁺⁺ and Er⁺⁺⁺.

### DETAILED DESCRIPTION OF THE INVENTION

The present invention relates to a process of inhibiting proliferation of neoplastic cells in a host, said process comprising administering to a host:
(a) a zinc reagent; and
(b) a compound of Formula I 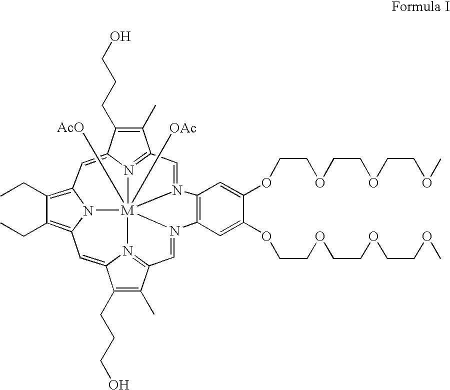
   wherein M is selected from Gd⁺⁺⁺, Eu⁺⁺⁺, Tb⁺⁺⁺, Dy⁺⁺, Ho⁺⁺⁺ and Er⁺⁺⁺.

A preferred embodiment provides a process wherein the zinc reagent is selected from zinc acetate, zinc chloride, zinc citrate, zinc lactate, and zinc complex of 1-hydroxypyridine-2-thione (ZnHPT, Formula II), wherein M in the compound of Formula I is selected from Gd⁺⁺⁺, Eu⁺⁺⁺ and Dy⁺⁺⁺.

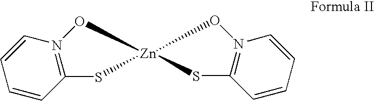

A preferred embodiment of the present invention provides a process for inhibiting proliferation of neoplastic cells in a host, said process comprising administering to a host:
(a) zinc acetate; and
(b) a compound of Formula I

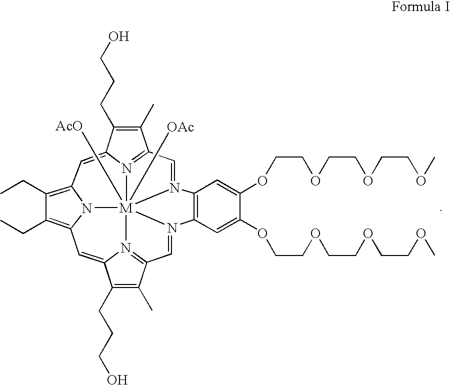

Yet another preferred embodiment provides a process for inhibiting proliferation of neoplastic cells in a host, said process comprising administering to a host:
(a) zinc acetate; and
(b) a compound of Formula I

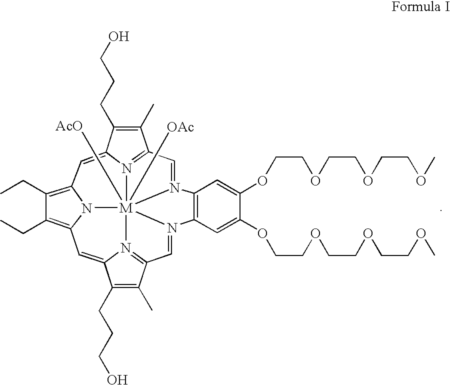

Yet another preferred embodiment provides a process for inhibiting proliferation of neoplastic cells in a host, said process comprising administering to a host:
(a) zinc acetate; and
(b) a compound of Formula I

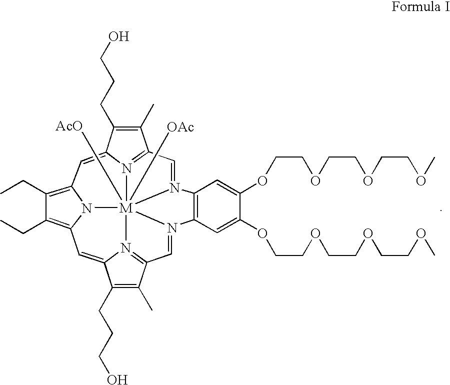

It is understood that the present invention can be practiced by first administering the zinc reagent, such as zinc acetate, followed by the compound of Formula I or vice versa or simultaneously. Similarly it is understood that a variety of zinc reagents can be used. Illustrative examples of the zinc reagent are zinc acetate, zinc chloride, zinc citrate, zinc lactate and 1-hydroxypyridine-2-thione (ZnHPT).

### DETAILED DESCRIPTION OF THE DRAWINGS

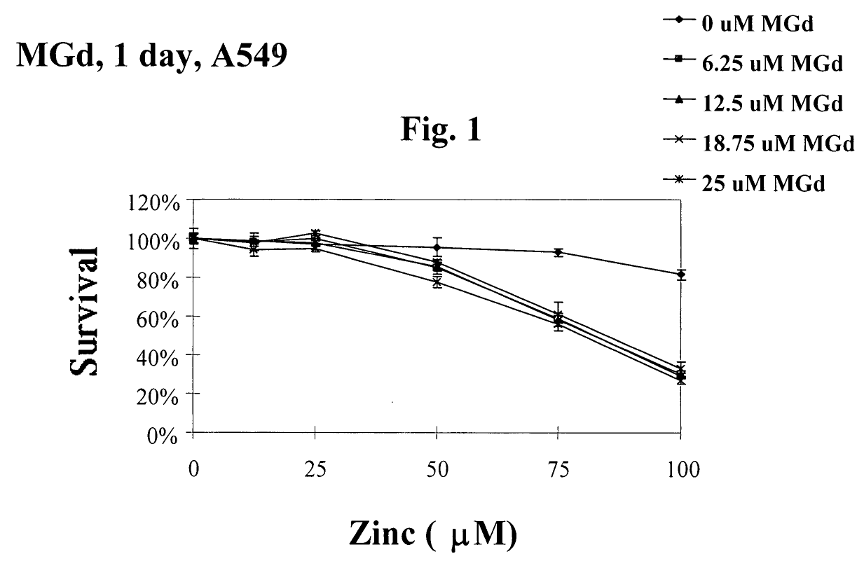

FIG. 1 shows the effect of a compound of Formula I, wherein M=Gd⁺⁺⁺ (MGd), and zinc on A549 cells after one day. As can be seen, MGd by itself has no effect on the A549 cells and zinc by itself (at 100 μM) has no significant effect on the A549 cells.

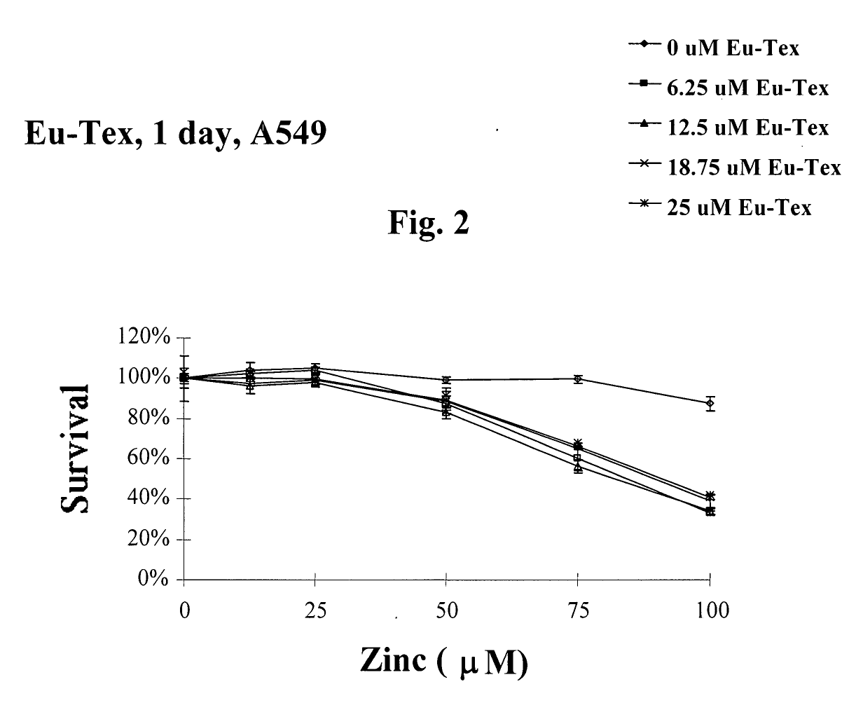

FIG. 2 shows the effect of a compound of Formula I, wherein M=Eu⁺⁺⁺ (Eu-Tex), and zinc on A549 cells after one day. As can be seen, Eu-Tex by itself has no effect on the A549 cells and zinc by itself (at 100 μM) has no significant effect on the A549 cells.

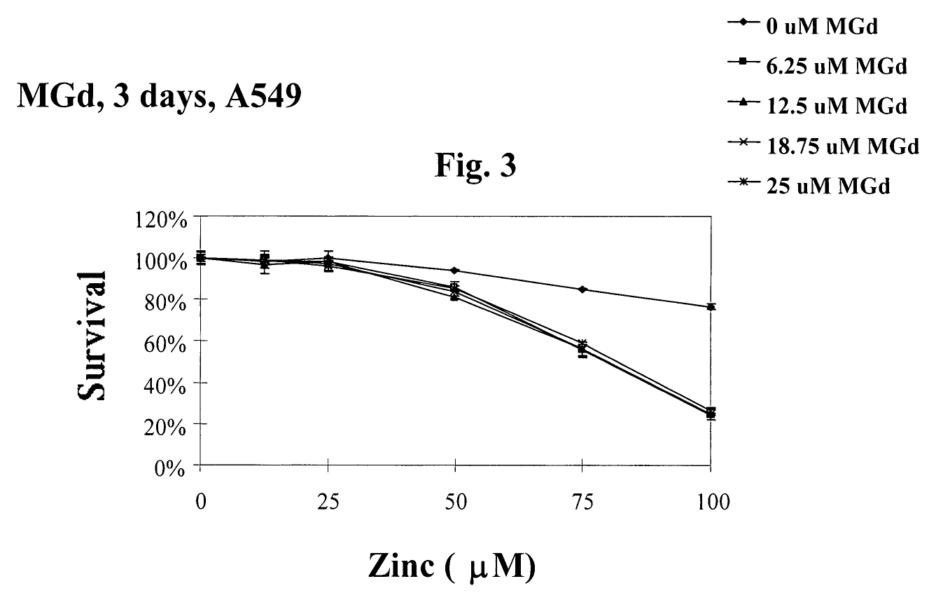

FIG. 3 shows the effect of a compound of Formula I, wherein M=Gd⁺⁺⁺ (MGd), and zinc on A549 cells after three days. As can be seen, MGd by itself has no effect on the A549 cells and zinc by itself (at 100 μM) has no significant effect on the A549 cells.

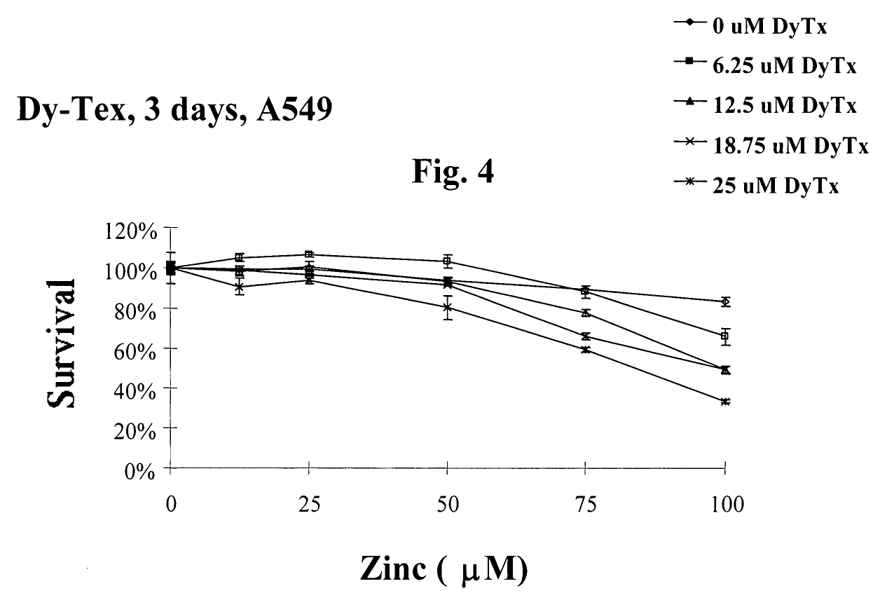

FIG. 4 shows the effect of a compound of Formula I, wherein M=Dy (Dy-Tex), and zinc on A549 cells after three days. As can be seen, Dy-Tex by itself has no effect on the A549 cells and zinc by itself (at 100 μM) has no significant effect on the A549 cells.

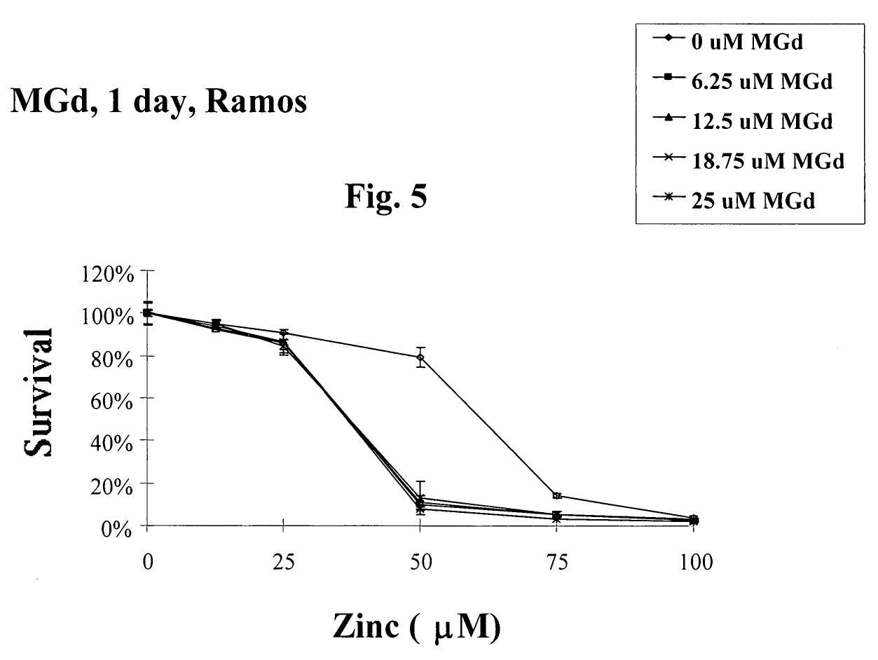

FIG. 5 shows the effect of MGd and zinc on Ramos cells after one day of treatment.

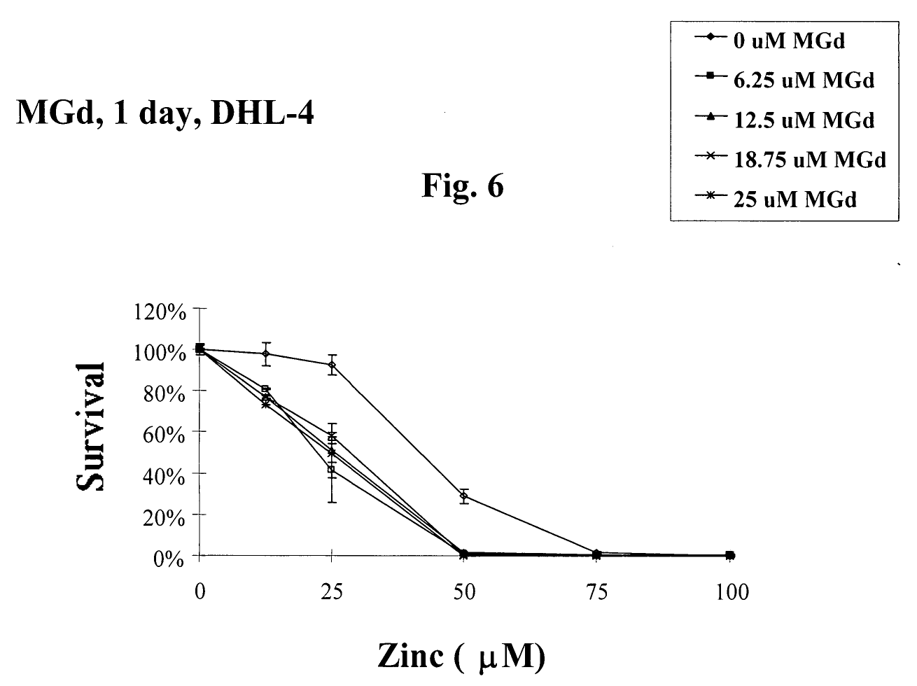

FIG. 6 shows the effect of MGd and zinc on DHL-4 cells after one day of treatment.

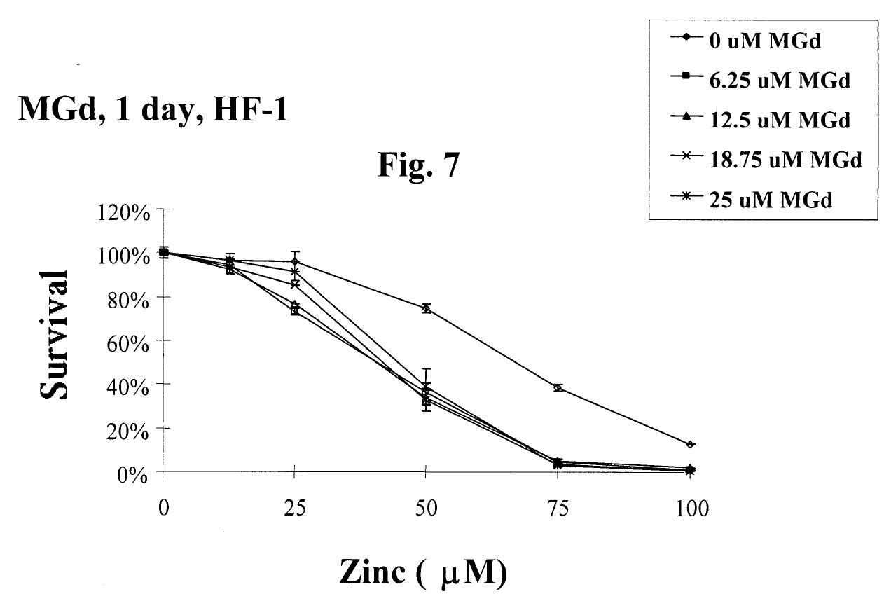

FIG. 7 shows the effect of HF-1 cells after one day of treatment.

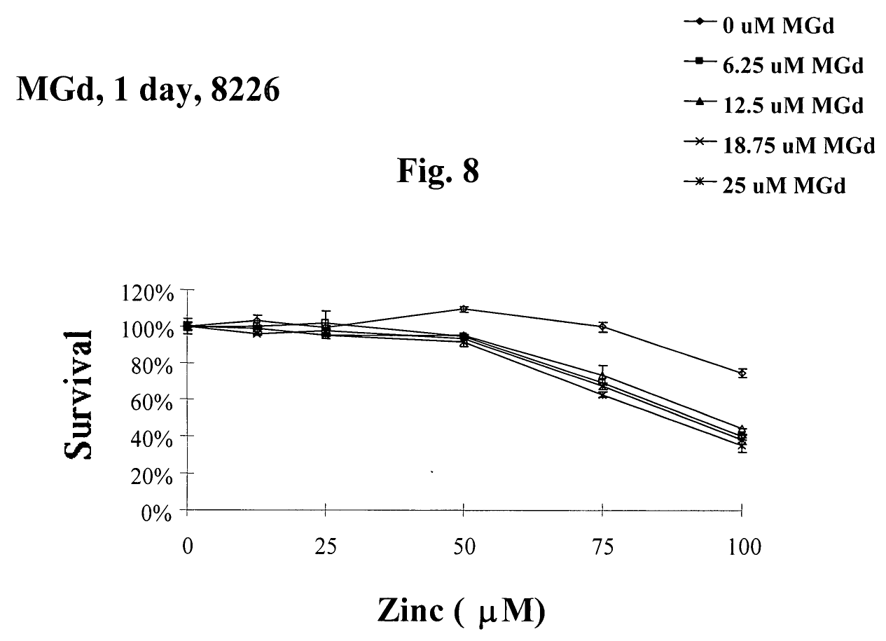

FIG. 8 shows the effect of MGd and zinc on 8226 cells after one day of treatment.

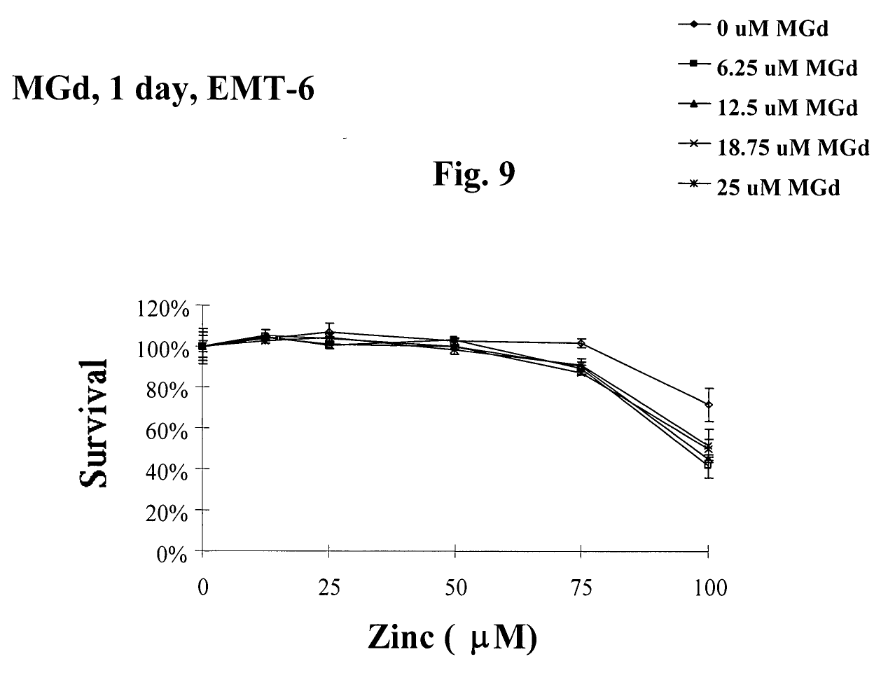

FIG. 9 shows the effect of MGd and zinc on EMT-6 cells after one day of treatment.

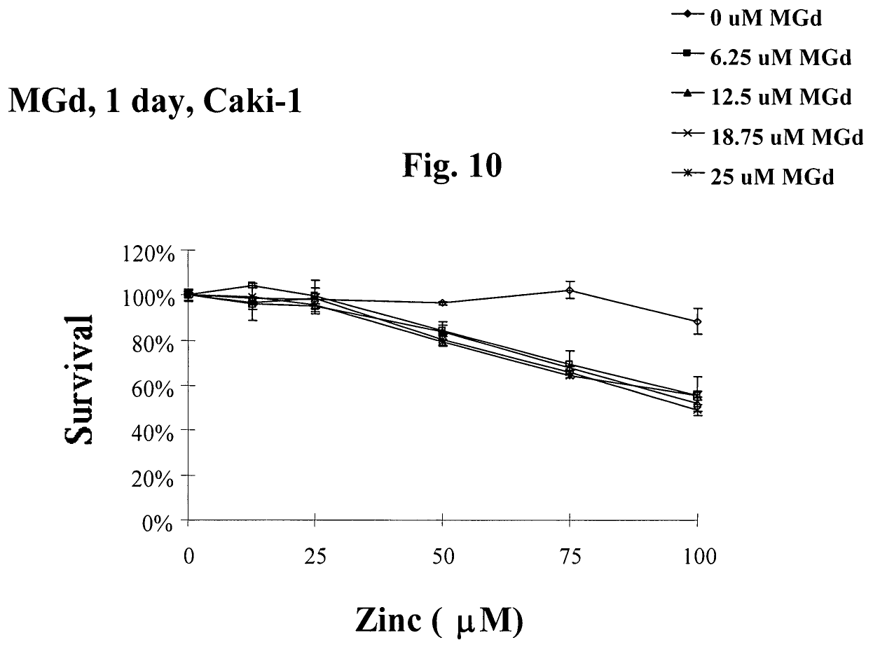

FIG. 10 shows the effect of MGd and zinc on Caki-1 cells after one day of treatment.

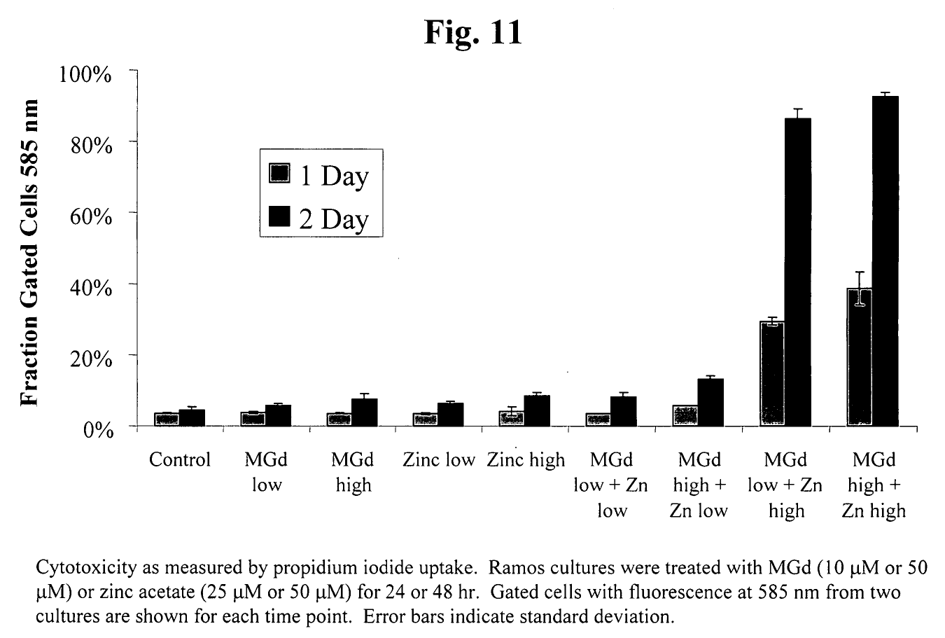

FIG. 11 shows the cytotoxicity as measured by propidium iodide uptake. Ramos cultures were treated with MGd (10 μM or 50 μM) or zinc acetate (25 μM or 50 μM) for 24 or 48 hr. Gated cells with fluorescence at 585 nm from two cultures are shown for each time point. Error bars indicate standard deviation.

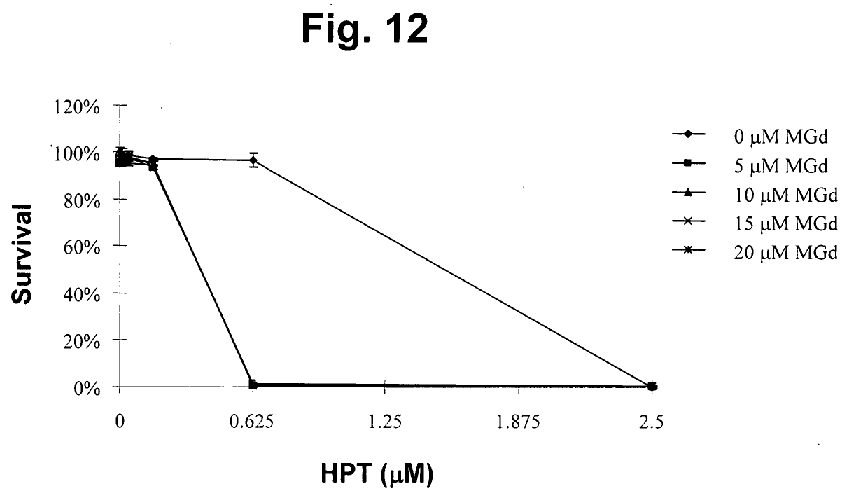

FIG. 12 shows the effect of a compound of Formula I, wherein M=Gd⁺⁺⁺ (MGd), and ZnHPT (formed in situ from zinc acetate and 1-hydroxypyridine-2-thione) on A549 cells after one day. As can be seen, MGd by itself has no effect on the A549 cells and ZnHPT by itself (at 0.625 μM) has no significant effect on the A549 cells.

### EXPERIMENTAL

The anti-proliferative activity of zinc and texaphyrin combinations was assessed using a microtiter plate format (FIGS. 1-11). Human lymphoma (DHL-4, HF-1, Ramos), myeloma (8226), renal carcinoma (Caki-1), lung carcinoma (A549), or murine mammary (EMT-6) cells were cultured in microplates containing RPMI 1640 medium supplemented with 10% fetal bovine serum. A549 or other adherent cells (Caki-1, EMT-6) were allowed to adhere to 96-well microtiter plates (2000 cells per well, Rows B-H) overnight. Suspension cell lines (DHL-4, HF-1, Ramos, and 8226) were added to V-bottom microplates at 40,000 cells/well. Zinc (0-100 μM) and 0-25 μM Formula I compond were added for 24-72 hr. Formula I compound M=Gd⁺⁺⁺ and zinc acetate (99.99%, Aldrich Chemical, St. Louis, Mo.) were formulated as 2 mM stock solutions in 5% aqueous mannitol. Final mannitol concentration was 0.31% in all wells. The plates were incubated at 37° C. under a 5% CO₂/95% air atmosphere. Medium was exchanged and proliferation was assessed using a tetrazole (MTT) reduction assay at the end of 3 days (Mosmann, T., J. Immunol. Methods, 65: 55-63 (1983)). In brief, 20 μL MTT dye (Sigma Chemical, St. Louis, Mo., catalogue no. M 2128, 5 mg/mL solution in phosphate buffered saline) was added to cells in medium to give a final volume of 200 μL. The plates were incubated at 37° C. for ca. 2 hours, whereupon the medium was removed and isopropyl alcohol (100 μL/well) was added. Microtiter plates were vortexed for ca. 3 minutes to dissolve MTT formazan, and then analyzed on a micro plate reader at 560-650 nm. The average absorption in the absence of cells (Row A) was subtracted from each well as background absorbance. Plate absorbances were normalized to the average of wells containing no zinc or texaphyrin. Data for each concentration of a compound of Formula I and zinc is the average of 2 wells. In other experiments (FIG. 11), Ramos cells grown in T-25 flasks were treated with a compound of Formula I (where M=Gd) (10 or 50 μM) and zinc (25 or 50 μM) for 1-2 days, Coulter counted, and analyzed by flow cytometry using propidium iodide. In other experiments (FIG. 12), A549 cells grown in microtiter plates at plateau phase were treated with 1-hydroxypyridine-2-thione (0-10 μM), MGd (0-20 μM), and zinc (50 μM). Plates were incubated at 37° C. for 24 hr, then analyzed using MTT as above.

Treatment with 6.25 μM or higher MGd inhibited the proliferation in the presence of zinc in all cell lines tested. A compound of Formula I alone had no effect on proliferation at the concentrations tested. In Ramos cells, a compound of Formula I (where M=Gd) (10 or 50 μM) and zinc (50 μM) increased cytotoxicity as assessed by flow cytometry after staining with propidium iodide. Zinc or a compound of Formula I (where M=Gd) alone did not increase cytotoxicity. The cytotoxic dose of the zinc complex of 1-hydroxypyridine-2-thione was lowered by a compound of formula 1. In summary, compounds of Formula I in combination with zinc inhibits cancer cell proliferation and causes cytotoxicity.

Compound Synthesis

Compounds of Formula I can be synthesized by procedures of Sessler et al., as detailed in U.S. Pat. Nos. 5,162,509; 5,252,720; and 5,801,229, the contents of which are incorporated herein by reference.

Abbreviations

MTT: 3-(4,5-dimethylthiazol-2-yl)-2,5-diphenyltetrazolium bromide
ZnHPT: Zinc complex of 1-hydroxypyridine-2-thione
HPT: 1-hydroxypyridine-2-thione

1. A process for inhibiting proliferation of neoplastic cells in a host, said process comprising administering to the host:
(a) a zinc reagent; and
(b) a compound of Formula I 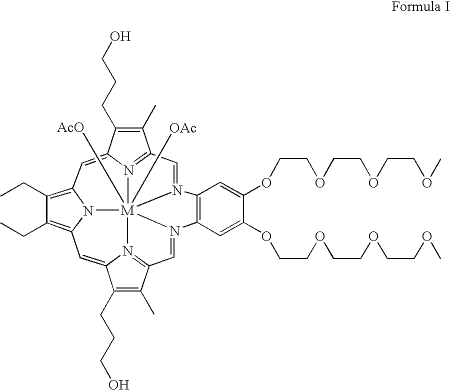
wherein M is selected from Gd⁺⁺⁺, Eu⁺⁺⁺, Tb⁺⁺⁺, Dy⁺⁺, Ho⁺⁺⁺ and Er⁺⁺⁺.

2. A process of claim 1 wherein the zinc reagent is selected from zinc acetate, zinc chloride, zinc citrate, zinc lactate, and zinc complex of 1-hydroxypyridine-2-thione (ZnHPT).

3. A process of claim 2 wherein M in the compound of Formula I is selected from Gd⁺⁺⁺, Eu⁺⁺⁺ and Dy⁺⁺⁺.

4. A process for inhibiting proliferation of neoplastic cells in a host, said process comprising administering to the host:
(a) zinc acetate; and
(b) a compound of Formula I

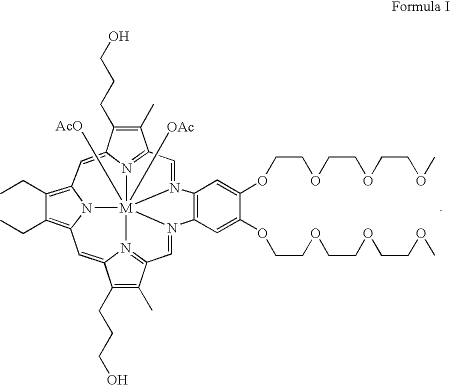

5. A process for inhibiting proliferation of neoplastic cells in a host, said process comprising administering to the host:
(a) zinc acetate; and
(b) a compound of Formula I

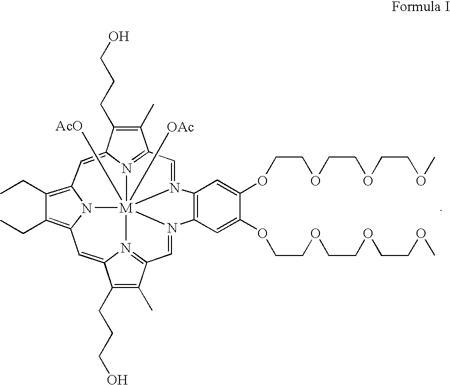

6. A process for inhibiting proliferation of neoplastic cells in a host, said process comprising administering to the host:
(a) zinc acetate; and
(b) a compound of Formula I [chemical structure]
